(19) United States
(12) Patent Application Publication
(10) Pub. No.: US 2020/0000646 A1
(43) Pub. Date: Jan. 2, 2020

(54) ABSORBENT ARTICLE

(71) Applicant: J2LFA CO., LTD., Seoul (KR)

(72) Inventor: Woo-Bok LEE, Seoul (KR)

(73) Assignee: J2LFA CO., LTD., Seoul (KR)

(21) Appl. No.: 16/247,848

(22) Filed: Jan. 15, 2019

(30) Foreign Application Priority Data
Jun. 27, 2018 (KR) 20-2018-0002931

(51) Int. Cl. A61F 13/472; A61F 13/15; A61F 13/511; A61F 13/53

(52) U.S. Cl. CPC A61F 13/472; A61F 13/15203; A61F 13/5616; A61F 13/53; A61F 2013/530007; A61F 13/511

(57) ABSTRACT

An absorbent article according to the present invention may comprise an absorbent pad 12, which includes: a wood pulp web 12b and a cotton-based nonwoven fabric 12a laminated on the wood pulp web 12b, wherein the nonwoven fabric 12a includes a valley part continuously formed on a surface thereof in a length direction and arranged at a predetermined interval in a width direction thereof. The absorbent article manufactured according to the present invention includes the absorbent pad layer 12 consisting of the cotton-based nonwoven fabric 12a, wherein the nonwoven fabric 12a includes the valley part continuously formed in the length direction and arranged at a predetermined interval in the width direction on the surface of the nonwoven fabric 12a in order to form channels C in the length direction of the absorbent pad 12, thereby effectively preventing leakage of the menstrual fluid or urine. Further, the absorbent article according to the present invention includes the wood pulp web 12b as well as the cotton-based nonwoven fabric 12a in the absorbent pad 12, thus to rapidly absorb the menstrual fluid or urine and safely keep the same therein.

### CROSS-REFERENCE TO RELATED APPLICATION

Pursuant to 35 U.S.C. § 119(a), this application claims the benefit of earlier filing date and right of priority to Korean Utility model Application No. 20-2018-0002931, filed on Jun. 27, 2018 in the Korean Intellectual Property Office, the entire disclosure of which is incorporated herein by reference.

### BACKGROUND OF THE INVENTION

### 1. Field of the Invention

The present invention relates to an absorbent article, and more particularly, to an absorbent article for effectively preventing leakage of menstrual fluid or urine in a width direction of the absorbent article, which can effectively prevent diverse female diseases, has excellent air-permeability, touch feel and comfortable fit, and can safely absorb/keep the menstrual fluid or urine in the absorbent article.

Hereinafter, in the present disclosure, a cotton cultivated using natural cotton seeds not subjected to a genetically modified organism (GMO) process without application of a compound fertilizer, insecticides and agricultural chemicals is referred to as an “organic cotton,” and a sanitary napkin and a diaper are referred to as an “absorbent article” as a whole.

### 2. Description of the Related Art

An absorbent article may include, for example, a sanitary napkin which is an absorbent pad for absorbing and containing secretion of a woman such as menstrual fluid therein. Generally, a conventional sanitary napkin includes a top cover layer, an absorbent pad and a bottom cover layer, which are laminated in this order, and has wing parts formed at both lateral sides of the sanitary napkin. The top cover layer directly comes in contact with the skin and serves to pass the menstrual fluid or vaginal discharge toward the absorbent pad or the like. The absorbent pad serves to directly absorb and keeps the menstrual fluid therein. Further, the bottom cover layer serves as a water-proof layer to prevent the menstrual fluid from being leaked or escaped to an outside.

As an example of the conventional sanitary napkins, as described in Korean Utility Model Registration No. 20-0305833, a sanitary napkin, which includes an absorbent pad formed of a synthetic fiber-based nonwoven fabric, pulp and a polymer absorbent, a top cover layer (i.e., a top sheet) formed of another synthetic fiber-based nonwoven fabric such as polypropylene, has been mainly used. However, the polymer absorbent and the synthetic fiber-based nonwoven fabric have caused problems such as degradation in air-permeability and touch feel, discomfort on wearing, etc. Further, a menstrual fluid leakage preventing line is not reliably formed, thereby it is not possible to effectively prevent the menstrual fluid from being leaked.

Another example of the conventional sanitary napkins, a sanitary napkin, which has a plurality of lines for preventing leakage of menstrual fluid (‘leakage prevention line’) formed of recesses along an outline of the absorbent pad, has been used. However, it is difficult for such a leakage prevention line to effectively prevent the menstrual fluid from being leaked in a width direction of the absorbent pad.

### SUMMARY OF THE INVENTION

An object of the present invention is to provide an absorbent article that may effectively prevent diverse female diseases, while having comfortable fit and touch feel and excellent air-permeability, and may rapidly absorb menstrual fluid or urine and safely keep the same therein, as well as may effectively block leakage of the menstrual fluid or urine.

In order to achieve the above object, the absorbent article manufactured according to the present invention may include an absorbent pad 12, which serves to absorb and keep menstrual fluid or urine therein and consists of a wood pulp web 12b and a cotton-based nonwoven fabric 12a laminated on the wood pulp web 12b, wherein the nonwoven fabric 12a includes a valley part continuously formed on a surface thereof in a length direction and arranged at a predetermined interval in a width direction thereof.

The absorbent article manufactured according to the present invention includes the absorbent pad layer 12 consisting of the cotton-based nonwoven fabric 12a, wherein the nonwoven fabric 12a includes the valley part continuously formed in the length direction and arranged at a predetermined interval in the width direction on the surface of the nonwoven fabric 12a in order to form channels C in the length direction of the absorbent pad 12, thereby effectively preventing leakage of the menstrual fluid or urine.

Further, the absorbent article according to the present invention includes the wood pulp web 12b as well as the cotton-based nonwoven fabric 12a in the absorbent pad 12, thus to rapidly absorb the menstrual fluid or urine and safely keep the same therein.

Further, according to the present invention, the nonwoven fabric 12a included in the absorbent pad 12 is formed of a cotton material, thus to effectively prevent diverse female diseases and ensure excellent air-permeability, touch feel and comfortable fit.

### BRIEF DESCRIPTION OF THE DRAWINGS

The above and other objects, features and other advantages of the present invention will be more clearly understood from the following detailed description taken in conjunction with the accompanying drawings, in which:

FIG. 1 is a cross-sectional view illustrating an absorbent pad 12 of the present invention;

FIG. 2 is a plan view illustrating a sanitary napkin according to an embodiment of the present invention; and

FIG. 3 is a cross-sectional view illustrating a straight type main body 10 in the sanitary napkin shown in FIG. 2, taken on line A-A′ in FIG. 2.

### DETAILED DESCRIPTION OF THE INVENTION

Hereinafter, the present invention will be described in detail with reference to the accompanying drawings.

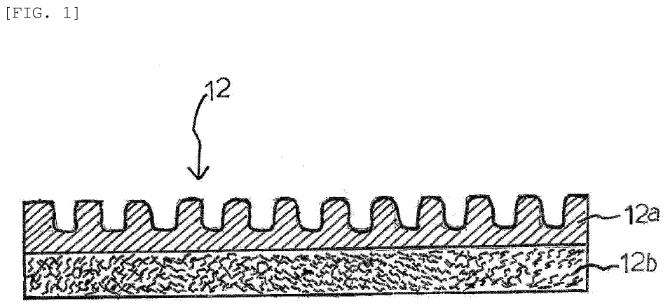

As shown in FIG. 1, an absorbent article of the present invention may include an absorbent pad 12 consisting of a wood pulp web 12b and a cotton-based nonwoven fabric 12a laminated on the wood pulp web 12b, wherein the nonwoven fabric 12a includes a valley part continuously formed on a surface thereof in a length direction and arranged at a predetermined interval in a width direction thereof.

The absorbent article described herein may be a sanitary napkin, a diaper, etc.

The nonwoven fabric 12a included in the absorbent pad 12 may made of cotton, preferably, organic cotton. Preferably, the nonwoven fabric 12a is a pressed nonwoven fabric form fabricated by a compression process, so as to prevent leakage of menstrual fluid or urine in a width direction of the absorbent article.

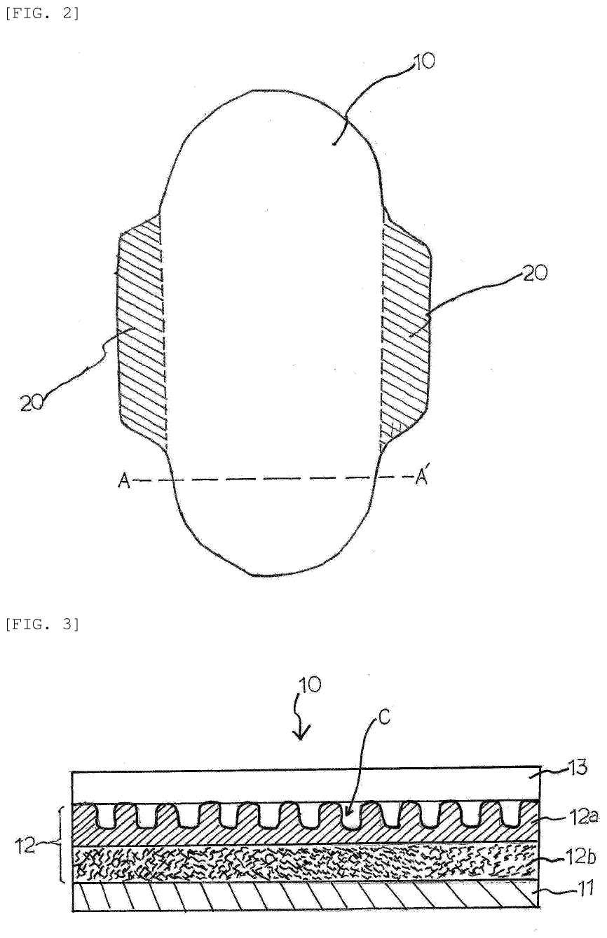

As an embodiment of the absorbent article, a sanitary napkin according to present invention may include a straight type main body 10 and wing parts 20 formed on both sides of the straight type main body 10, respectively, as shown in FIG. 2.

The straight type main body 10 may include: (i) a bottom cover layer 11 made of a water-proof film; (ii) an absorbent pad 12 consisting of a wood pump web 12b and a cotton-based nonwoven fabric 12a laminated on the wood pulp web 12b; and (iii) a top cover layer 13 made of cotton and formed in a nonwoven fabric form.

As shown in FIGS. 1 and 3, the nonwoven fabric 12a included in the absorbent pad 12 may have a structure in which a valley part is continuously formed on a surface thereof in a length direction and arranged at a predetermined interval in a width direction thereof.

As shown in FIG. 3, channels C may be formed by the valley part between the nonwoven fabric 12a included in the absorbent pad 12 and the top cover layer 11. Therefore, the menstrual fluid does not leak in the width direction but flows in the length direction of the absorbent pad 12 to thus be absorbed therein.

The nonwoven fabric 12a is preferably a pressed nonwoven fabric fabricated by inter-mingling a web, which is a laminate of cotton mono fabrics, through needle-punching, followed by compression using a calendaring roller or the like.

The bottom cover layer 11 may be a synthetic resin film or a bio-degradable film, and the bio-degradable film is preferably used to minimize environmental contamination in consideration of disposal after use.

The nonwoven fabric 12a included in the absorbent pad 20 and the top cover layer 13 are made of a nonwoven fabric material including cotton only, thereby achieving excellent effects in preventing female diseases and excellent air-permeability, touch feel and comfortable fit, simultaneously.

According to the present invention, the valley part is continuously formed on the surface of the cotton-based nonwoven fabric 12a included in the absorbent pad 12 in the length direction and arranged at a predetermined interval in the width direction thereof, thus to form channels C in the length direction of the absorbent pad 12. As a result, leakage of the menstrual fluid in the width direction of the absorbent article may be effectively prevented.

Further, the absorbent pad 12 of the present invention includes the wood pulp web 12b as well as the cotton-based nonwoven fabric 12a, thereby rapidly absorbing the menstrual fluid and safely keeping the same therein.

### DESCRIPTION OF REFERENCE NUMERALS

12: Absorbent pad
12a: Nonwoven fabric included the absorbent pad
12b: Wood pulp web included the absorbent pad
C: Channel
10: Straight type main body of sanitary napkin
20: Wing part of sanitary napkin
11: Bottom cover layer
13: Top cover layer

What is claimed is:

1. An absorbent article comprising an absorbent pad 12, which includes: a wood pulp web 12b and a cotton-based nonwoven fabric 12a laminated on the wood pulp web 12b,
wherein the nonwoven fabric 12a includes a valley part continuously formed on a surface thereof in a length direction and arranged at a predetermined interval in a width direction thereof.

2. The absorbent article according to claim 1, wherein the absorbent article is one selected from a sanitary napkin and a diaper.

3. The absorbent article according to claim 1, wherein the nonwoven fabric 12a included in the absorbent pad 12 is made of organic cotton.

4. The absorbent article according to claim 1, wherein the nonwoven fabric 12a included in the absorbent pad 12 is a pressed nonwoven fabric fabricated by a compression process using a calendaring roller.

5. The absorbent article according to claim 2, wherein the sanitary napkin comprises a straight type main body 10 and wing parts 20 formed on both sides of the straight type main body 10,
wherein the straight type main body 10 includes: (i) a bottom cover layer 11 made of a water-proof film; (ii) an absorbent pad 12 consisting of a wood pump web 12b and a cotton-based nonwoven fabric 12a laminated on the wood pulp web 12b; and (iii) a top cover layer 13 made of cotton and formed in a nonwoven fabric form, and
the nonwoven fabric 12a includes a valley part continuously formed on a surface thereof in a length direction and arranged at a predetermined interval in a width direction thereof.

6. The absorbent article according to claim 5, wherein each of the wing parts 20 includes: (i) a bottom cover layer made of a water-proof film; and (ii) a top cover layer laminated on the bottom cover layer, made of cotton and formed in a nonwoven fabric form.
